(19) United States
(12) Patent Application Publication
(10) Pub. No.: US 2003/0039622 A1
(43) Pub. Date: Feb. 27, 2003

(54) Concept of incorporating glacier silt into any cleaning composition for human skin

(76) Inventor: Stephanie Lynn Varner, Juneau, AK (US)

Correspondence Address: Stephanie Lynn Varner, PO Box 35762, Juneau, AK, 99803, US

(21) Appl. No.: 10/218,319

(22) Filed: Aug. 13, 2002

Related U.S. Application Data
(60) Provisional application No. 60/313,708, filed on Aug. 21, 2001.

(51) Int. Cl. A61K 7/06; C11D 1/00; A61K 7/50

(52) U.S. Cl. 424/70.1; 510/130

(57) ABSTRACT

The concept of incorporating glacier silt into any cleaning composition for human skin has not been previously patented to my knowledge.

### CROSS-REFERENCE TO RELATED APPLICATIONS

This application refers to Provisional Application No. 60/313708 filed on Aug. 21, 2001.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF INVENTION

This invention pertains to US Patent Classification 510, (“Cleaning Compositions for Solid Surfaces, Auxiliary Compositions Therefore, or Processes of Preparing the Compositions”), specifically, subclass 139 (“With particulate scrubbing or abrasive component”), under subclass 130 (“For Human Skin”).

### BRIEF SUMMARY OF THE INVENTION

The concept of incorporating glacier silt into any cleaning composition or auxiliary composition to be used for human skin is claimed as my invention. Glacier silt in it's naturally occurring consistency is very fine and acts as an almost undetectable pumice.

### BRIEF DESCRIPTION OF THE SEVERAL VIEWS OF THE DRAWING

Not Applicable

### DETAILED DESCRIPTION OF THE INVENTION

Glacier silt is harvested in it's natural form from areas previously occupied by glaciers. The silt is then heated at 400 degrees, sifted for a uniform consistency, and blended into cleaning compositions used for human skin. Applied to human skin, the composition acts as a mild pumice. Glacier silt is distinctive from other abrasives or pumices because the silt, in it's natural form, is made up of very fine particles and is essentially undetectable when being used.

What I claim as my invention is:

1. The concept of incorporating glacier silt into any cleaning composition for human skin.
